(19) United States
(12) Patent Application Publication
(10) Pub. No.: US 2010/0272799 A1
(43) Pub. Date: Oct. 28, 2010

(54) COATING TECHNOLOGY TO PRODUCE A COMBINATION SOLID DOSAGE FORM

(76) Inventor: Hemant N. Joshi, Parsippany, NJ (US)

Correspondence Address: HEMANT N. JOSHI, 41 LEAH WAY, PARSIPPANY, NJ, 07054, US

(21) Appl. No.: 12/430,870

(22) Filed: Apr. 27, 2009

(51) Int. Cl. A61K 9/28; A61K 9/14; A61K 9/48

(52) U.S. Cl. 424/463; 424/484; 424/474

(57) ABSTRACT

The present invention claims the usage of coating technology to produce multi-segmented pharmaceutical dosage forms for a combination drug therapy. The core matrix containing a drug is coated with drug containing solutions or suspensions. The separation of different drugs in the core and various coating segments prevents drug-drug interaction and improve drug stability, provide desired release rates for different drugs in the dosage form. The dosage form may release drugs in different parts of the gastrointestinal tract.

### BACKGROUND OF THE INVENTION

Many good drugs have been invented in the recent years and serving our community very well. However, it is not very easy to invent new drug molecules for various reasons and many a times, it is not necessary to invent new molecules for some known diseases. Scientists are paying attention to the combination drug therapy in which more than one active moiety is incorporated in a dosage form. The active moieties may have synergistic actions or may help reduce the side effects. Combination drug therapy is expected to continue to be the part of future drug and dosage form development. Anti-cancer and Anti-AIDs drugs are frequently administered in combination. Combination drug therapy is also used for treating other diseases too. In the U.S. Pat. No. 6,835,728, the combination of mirtazapine and gepirone was claimed to be better to treat depression. U.S. Pat. No. 6,809,120 claimed that administering propargylamine before, after and concurrently with an anti-neoplastic drug increased the selectivity and effectiveness of the said drug. In the U.S. Pat. No. 6,960,577, a combination therapy of Olanzapine and Fluoxetine to treat refractory depression was disclosed. U.S. Pat. No. 6,942,876 claimed that a combination therapy of antiepileptic compounds that demonstrated pain-alleviating property and compounds from a group consisting of analgesics, NMDA receptor agonists, and NSAIDs decreased the frequency and severity of pain and reduced the side effects.

Combination drug therapy also allows avoidance of taking multiple tablets/capsules per day, savings on the co-payment for different medicines and assurance of patient-compliance to drug therapies.

It is important to prove that different drugs combined in the same dosage form are stable during manufacture and storage and should not interact physically or chemically with each other. In the U.S. Pat. No. 7,226,614, tablets of cetirizine and pseudoephedrine were made with drugs in two different segments to avoid drug-drug interaction. The pharmacokinetic profiles of each drug were the same as the pharmacokinetic profiles from tablets of individual drugs. In the combination dosage form, each drug should show the desired release rate from the dosage form to produce the desired rate and extent of absorption upon oral administration or as an implant. This is important mainly for drugs with low solubility and high/low permeability. Drugs when mixed together in the same matrix, may interact physically or chemically affecting their stability in the dosage form. As a result, it may affect their potency as well as pharmacological effectiveness.

Coating of pharmaceutical dosage forms such as tablets, capsules etc. is known to everyone working in the pharmaceutical field. The coating may have various functions such as, providing color to the dosage form, taste masking (U.S. Pat. No. 5,728,403), as an aid for swallowing, controlled/modified drug release, and enteric coating. Polymers are widely used in the film coating. In recent news item, MIT researcher, Dr. Paula Hammand and her colleagues developed “Smart” drug coatings using new types of polymers resulting in desired drug release rates. U.S. Pat. No. 6,908,626 described a composition where different release rates were obtained for the poorly soluble active ingredient using nano- and micro-particles. US patent application 20070071821 used a mixture of shellac and alginates to produce an enteric-coated oral dosage form. In another patent (US patent application 20050142097), two drugs with the same charge were conjugated with a resin and the particles were coated with various polymers such as ethyl cellulose and methylcellulose. Medical devices are often coated with polymer (U.S. Pat. No. 7,056,550). In the U.S. Pat. No. 6,746,686, the anionic drug was conjugated with the polymer coating on the stent or an implant having pendant cationic or zwitterionic groups.

Coating of tablets, capsules, and pills is performed mainly in the coating pan. In recent years, small pills, beads or particles can be coated in a fluid bed dryer. Hot air is mainly used to dry the coated solution. Joshi et al. proposed the use of microwave drying in film coating (Int. J. Pharm. 51: 19-25, 1989). US Microwaves, Inc., Santa Clara, Calif. provides custom film coating services using microwaves for ceramics, glass, quartz, etc. Malinger et al described the usage of microwave drying in coating articles (US patent application 20070154639). Vacuum drying may also be used to evaporate solvent from the coats.

### SUMMARY OF THE INVENTION

The present invention proposes the use of coating technology to produce drug-loaded coating segments on tablets, pills, beads and powder dosage forms for a combination drug therapy. These drug delivery systems can be used for oral, nasal, pulmonary, and implantable routes of administration of medicaments. The invention describes a multi-segmented dosage form in which the core region is either a drug particle, or a conventional pharmaceutical dosage form such as a tablet, capsule, bead, or a pill. The core region contains preferably one kind of drug. Normally each coating segment contains one drug. The combination dosage form contains 1 to 10 drugs, preferably 1 to 5 and more preferably 1 to 3 drugs. The number of drug-loaded coating segments in the dosage form could be 1 to 6, preferably 1 to 4 and most preferably, 1 to 2, respectively assuming one drug incorporated in the core matrix.

The drug-coatings in the dosage form are achieved using a solution, suspension or a gel formulation of the drug. Dividing layers are applied underneath or above the drug-loaded coating segments to allow physical separation of different drugs. The dividing layers are made up with commonly used polymers, sugars, shellac or gelatin and provide a sustained, controlled, modified, slow, thermally activated, or pH-activated release of drugs.

The combination dosage form may contain a drug in the core matrix for slow release and the same drug in the coating layer for an immediate release.

The dosage form containing multiple drugs in the core matrix and in the coating segments should have better drug stability profile and desired release rates for each drug.

The dosage form can be a tablet wherein micro-quantities of drug solutions containing different drugs, preferably 100 μL to 200 μL and more preferably 5 to 50 μL, are coated on the tablet surface as a spot or multiple spots. This tablet can then be coated with a protective or enteric-coated layer to achieve desired release.

### DETAILED DESCRIPTION

The key aspect of the present invention is to prepare multiple coat segments on the core using the coating technology and incorporate different drug components in some of the coats. The core region of the dosage form can be a drug particle or a conventional pharmaceutical dosage form such as a tablet, capsule, pill, bead or an implant. The core region can be prepared using commonly used excipients and employing a commonly used manufacturing process known to people working in the pharmaceutical field.

The central idea of the invention is to load different drugs in the same dosage form using the coating technology developing a unit dosage form with multiple drugs.

If the drug solution is coated directly on the core region, the solution will have tendency to penetrate into the matrix. The depth of penetration will depend upon the compositions of core region and/or the coating solution and the process. This may allow the interaction between the drug in the core and the drug in the coat. In order to avoid this, it is important to coat the core region with a dividing layer. The composition and thickness of the dividing layer will depend upon the desired function. If the function is merely the separation of drugs in the core matrix and the coat, a simple coating of sugar or a water-soluble polymer such as, hydroxypropyl methylcellulose may be sufficient. If one desires to have a controlled, slow or sustained release of the drug in the core matrix, a variety of pharmaceutically approved polymers can be used. For enteric coating, a different set of commonly known polymers is used. A large variety of polymers are available with different properties including film-forming nature, swelling, gel formation, water penetration etc.

A drug to be coated on the core region can be dissolved or suspended in a suitable solvent system to produce the coating formulation. The drug to be coated on the core region, if charged, can be ion-paired with an oppositely charged excipient—either a small molecule or a polymer. The drug can also be covalently bound to the polymeric matrix to form a polymeric prodrug. The intention is to attain a desired drug release rate from the dosage form. The drug may be dissolved in an emulsion or in a number of commonly available surfactant systems. These surfactants in these systems would also help the release of drug from the dosage form and may aid drug absorption.

It is important that the solvent can be evaporated easily during the coating operation using conventional system such as air or vacuum or fluid-bed drying. The amount of drug to be loaded can be varied based on the desired dose per unit.

The dosage form can be coated with additional layers, a sequence of dividing layer and the drug coat. During the coating operation, a precaution must be taken not to overheat the product to maintain the stability of drugs in the matrix and in the initial coat. The order of incorporation of drugs in the core matrix and coats may be varied based on the desired release sequence and rates can be altered using the correct dividing coat. This provides a better control to the formulator to obtain a desired pharmacokinetic profile.

It is possible that a drug is delivered in a controlled fashion from the core matrix and the same drug in the coat produces an immediate release. This may have a huge clinical significance.

The drugs in the coating segments may be in the solution or suspension form. The particle size of the drug in the suspension form will control the release rate if the drug has low aqueous solubility. The advent of nanotechnology may help produce a coating solution containing drug as nanoparticles. One can use microparticles or macroparticles in the suspension resulting into different release rates from the same dosage form.

In a system, core beads containing different drugs are coated with solutions containing different set of drugs. These beads are delivered in a capsule dosage form enabling to put together multiple drugs in the same dosage form. There could be many permutations and combinations of this kind of system and many types of polymeric solutions can be applied to achieve different release rates.

With the development of technology, very small volumes of liquids can be delivered (less than 100 μL). In a system, tiny droplets of drug solutions/suspensions are applied on a tablet surface. The solution on the tablet is dried immediately with a jet of hot air. A polymer layer can be applied to the tablet surface prior to or upon the addition of drug solutions or both producing a combination drug dosage form.

The coating technology is very important for potent drugs for two reasons. The amount of drug applied is limited in a coat. More importantly, the coat is applied in a solution/suspension form. Thus, there is no dusting of drug and the manufacturing personnel will be less exposed to the potent drugs.

The delivery systems described in this patent are intended for oral, nasal, pulmonary, and implantable routes of administration.

What is claimed is:

1. A drug delivery system containing a drug-loaded core matrix, drug-loaded coats and separating layers producing a combination drug dosage form.

2. A drug delivery system as in claim 1 wherein the core matrix is a drug particle, bead, pill, tablet, capsule or an implant.

3. A drug delivery system as in claim 2 wherein the different drugs are coated in bead matrices, which are further coated with different set of drugs and these beads are incorporated in a capsule dosage form.

4. A drug delivery system as in claim 1, which is intended for oral, nasal, pulmonary and implantable routes of administration.

5. The dosage form as in claim 1 wherein the drug coating is achieved using a solution, suspension or a gel formulation of the drug.

6. The coating suspension as in claim 5 wherein the drug is in the form of nanoparticles, microparticles or macroparticles resulting into different release rates.

7. The drug delivery system as described in claim 1 which contains 1 to 10 drugs, preferably 1 to 5 and more preferably 1 to 3 drugs.

8. The drug delivery system as in claim 1 wherein the number of drug-loaded coating segments are 1 to 6, preferably 1 to 4 and most preferably, 1 to 2.

9. The dosage form as in claim 1 wherein the drug containing coating segments are separated by one or two dividing layers made up with commonly used polymers, sugars or gelatin and serve a purpose of sustained, controlled, modified, slow, thermally-activated, or pH-activated release of drugs.

10. The dosage form as in claim 1 wherein the drug in the core matrix is intended for slow, sustained, or controlled release and the coating layer is intended for an immediate release of the same drug.

11. The dosage form as in claim 1 wherein the core matrix is a tablet and micro-quantities of drug solutions containing different drugs, preferably 100 μL to 200 μL and more preferably 5 to 50 μL, are coated on the tablet surface as a multiple spots followed by protective or enteric-coated layers.
